(19) United States
(12) Patent Application Publication
(10) Pub. No.: US 2025/0144430 A1
(43) Pub. Date: May 8, 2025

(54) CARDIAC PACING DEVICE AND DUAL-CHAMBER PACING SYSTEM

(71) Applicant: BIOTRONIK SE & CO. KG, Berlin (DE)

(72) Inventor: Andreas KUTSAM, Berlin (DE)

(73) Assignee: BIOTRONIK SE & CO. KG, Berlin (DE)

(21) Appl. No.: 18/833,671

(22) PCT Filed: Jan. 19, 2023
(86) PCT No.: PCT/EP2023/051237

§ 371 Date: Jul. 26, 2024

(30) Foreign Application Priority Data
Feb. 8, 2022 (EP) 22155533.7

(51) Int. Cl. A61N 1/365; A61N 1/368; A61N 1/375

(52) U.S. Cl. CPC A61N 1/36585; A61N 1/36578; A61N 1/368; A61N 1/37512; A61N 1/3756

(57) ABSTRACT

A cardiac pacing device for implantation in a patient's heart, wherein the cardiac pacing device comprises a processing unit for controlling a pacing signal generator and further comprises a detector configured to measure a time-dependent signal of an intrinsic atrial activity, wherein the processing unit is configured to detect a presence and absence of an intrinsic atrial event within a cardiac cycle based on the measured signal of the atrial activity within the respective cardiac cycle received from the detector, wherein the processing unit is configured to control the pacing signal generator to provide an electrical signal based on the presence and absence of an intrinsic atrial event. Further, a system comprising such first cardiac pacing device and a second cardiac pacing device is explained as well as respective operation methods.

### CROSS-REFERENCE TO RELATED APPLICATIONS

This application is the United States National Phase under 35 U.S.C. § 371 of PCT International Patent Application No. PCT/EP2023/051237, filed on Jan. 19, 2023, which claims the benefit of European Patent Application No. 22155533.7, filed on Feb. 8, 2022, the disclosures of which are hereby incorporated by reference herein in their entireties.

### TECHNICAL FIELD

The present invention is generally directed to a cardiac pacing device, an operation method of such device, a system comprising a first cardiac pacing device and a second cardiac pacing device, and an operation method of such system, a respective computer program product and computer readable data carrier.

### BACKGROUND

A cardiac pacing device such as a cardiac pacemaker (or artificial pacemaker) is a medical device that generates electrical pulses delivered by electrodes connected to or fixed at the pacemaker to cause the heart muscle chambers (i.e., the atria and/or the ventricles) to contract and therefore pump blood. By doing so this device replaces and/or regulates the function of the electrical conduction system of the heart. One purpose of a cardiac pacing device is to maintain an adequate heart rate (cardiac rate), either because the heart's natural pacemaker is not fast enough, or because there is a block in the heart's electrical conduction system. Modern pacemakers are externally programmable and allow a health care practitioner (HCP), such as a clinician, to select the optimal pacing mode(s) for individual patients.

A conventional pacemaker comprises a controlling and generator device comprising a processing unit and a power source external of the patient's heart and electrodes that are implanted within the heart's muscle. The electrodes are connected via leads and a header located at the device to the device. In most cases the device is implanted subcutaneously in the front of the chest in the region of the left or right shoulder. An implantable intra-cardiac pacemaker (also known as implantable leadless pacemaker—ILP) is a miniaturized pacemaker which is entirely implanted within a heart's ventricle (V) or atrium (A) of a patient. Alternative or additional functions of conventional or intra-cardiac pacemakers comprise providing other electrical or electromagnetic signals to the heart or its surrounding tissue and sensing electrical or electromagnetic signals (e.g., signals from electrical depolarization fields) or other physiological parameters of the heart and/or its surrounding tissue such as the intrinsic (i.e., the heart's natural) atrial contraction or the intrinsic (i.e., the heart's natural) ventricular contraction. Due to the highly restricted device size, an ILP has a small battery capacity.

An ILP may be operated in VDD pacing mode (i.e., a pacing mode in which the ventricle is stimulated according to the intrinsic atrial signal and AV conduction monitoring). In the VDD mode, the pacemaker synchronizes ventricular pacing with the intrinsic atrial timing by sensing when atrial contractions (i.e., the intrinsic atrial signals) occur. In an ILP that is implanted in the right ventricle, the atrial contraction information can be detected, but with less reliability and accuracy than in a dual chamber conventional pacemaker where there is a lead in the right atrium as well as the right ventricle.

However, there are circumstances in which a patient suffers from various cardiac arrhythmias that require different cardiac therapies. In such cases, a system of devices may be implanted comprising at least two cardiac pacing devices or units, wherein pacing may be provided within the atrium using the first device and within the ventricle using the second device.

U.S. Publication No. 2016/0067490 A1 discloses a dual-chamber, leadless pacing system comprising at least one atrial pacing device and at least one ventricular pacing device. To adjust the pacing rate in such system signals are transmitted from the atrial pacing device to the ventricular pacing device or from the ventricular pacing device to the atrial pacing device, wherein the respective pacing rate is adjusted based on the received signal. For that a separate communication unit of the atrial pacing device or the ventricular pacing device is provided that includes suitable hardware (e.g., an antenna), firmware, software or any combination thereof and consumes energy while communicating with the respective other pacing device.

As indicated above, the new dual chamber technology brings several challenges with it. Due to the size restrictions of intracardiac devices requirements for energy consumption and use of electronic components have increased drastically. Additionally, intracardiac devices that are intended to be implanted in the heart's atrium require specialized design considerations. The available volume for device placement is smaller compared to the ventricle, and the atrial tissue is significantly thinner compared to the ventricle. Further, the communication within the dual-chamber system may face problems concerning compatibility, in particular if different system members are provided by different suppliers/manufacturers or if a part of an intracardiac device (battery, communication unit) is exchanged during the service life of the respective device (e.g., in case of box-change at battery depletion).

Accordingly, there is the need for cardiac pacing devices which better cope with the requirements for energy consumption, fixation at the heart's tissue and available space and which ensure compatibility between the system members.

The present disclosure is directed toward overcoming one or more of the above-mentioned problems, though not necessarily limited to embodiments that do.

### SUMMARY

At least the above problem is solved by a cardiac pacing device comprising the features of claim 1, by a system comprising a first cardiac pacing device and a second cardiac pacing device comprising the features of claim 5, respective operation methods of such cardiac pacing device or system comprising the features of claims 8 and 12, respectively, as well as by a computer program product comprising the features of claim 14 and computer readable data carrier comprising the features of claim 15.

In particular, at least the above problem is solved by a cardiac pacing device for implantation in a patient's heart, wherein the cardiac pacing device comprises a processing unit for controlling a pacing signal generator and further comprises a detector configured to measure a time-dependent signal of an intrinsic atrial activity. The processing unit is configured to detect a presence and absence of an intrinsic atrial event (i.e., intrinsic atrial depolarization followed by atrial contraction, spontaneous p-wave) within a cardiac cycle based on the measured signal of the atrial activity within the respective cardiac cycle received from the detector. Further the processing unit is configured to control the pacing signal generator such that it provides a first electrical signal in case of absence of the intrinsic atrial event within the respective cardiac cycle and a second electrical signal in case of presence of the intrinsic atrial event within the respective cardiac cycle, wherein the second electrical signal is different from the first electrical signal.

The above defined cardiac pacing device further comprises at least one electrode electrically connected to the pacing signal generator, for example via a header, wherein the at least one electrode, e.g., two electrodes, is adjacent to the heart's tissue or implanted within the heart's tissue to be paced after implantation. The first electrical signal and the second electrical signal produced by the pacing signal generator are transmitted to the at least one electrode so that the first electrical signal and the second electrical signal are applied to the heart's tissue adjacent to the at least one electrode. Accordingly, the pacing device stimulates the myocardium by the first electrical (pacing) signal if the intrinsic atrial signal is not detected within the respective, actual cardiac cycle. Additionally, the cardiac pacing signal generator provides a second electrical signal within the respective cardiac cycle which is transmitted to the at least one electrode and applied to the heart by the at least one electrode. Accordingly, an electrical signal (e.g., a stimulation signal) is provided within each cardiac cycle, either a first electrical signal for atrial pacing if an atrial sense is absent or a second electrical signal. Thereby a (modified) AAT mode is realized, wherein the pacemaker's AAT mode refers to a mode in which the paced heart chamber is the atrium, the sensed chamber is the atrium, as well, and the response to sensing is characterized as “triggered” indicating that an electrical pulse (e.g., a stimulation) is provided once within each cardiac cycle. The pacing mode is characterized as “modified” because there are two different electrical signals provided, the first electrical signal and the second electrical signal, wherein the first electrical signal is supposed to successfully capture the myocardium and the second electrical signal is not supposed to capture the myocardium but to indicate to another device (a sensor or another cardiac pacing device) that there was an intrinsic atrial event within the respective cardiac cycle. The first and the second electrical signal may be detected by a sensor or another cardiac pacing device and thereby the information regarding the presence or absence of the intrinsic atrial signal may be detected, wherein the absence of an intrinsic atrial signal led to an electrical stimulus capturing the myocardium and the presence of an intrinsic atrial signal led to the triggered stimulus/electrical signal. Accordingly, a separate communication transmitting such information is not necessarily leading to reduced size of the cardiac pacing device and reduced energy consumption compared with the above described prior art so that the longevity of the cardiac pacing device is increased. Additionally, compatibility problems in connection with a separate communication channel transmitting such information are not there, anymore, since electrical signals of the heart may be detected by different detectors.

The cardiac pacing device may a defibrillator with pacing functionality or any other device with pacing functionality or an ILP, wherein each of the examples has the general structure and functionality as indicated above and below.

If the cardiac pacing device is an ILP one electrode may be located at a distal end of the ILP, close to a fixation member by which the ILP is fixed in the tissue of the patient's heart, for example within the inner tissue of a ventricle. A second electrode may be located at the proximal end of the ILP or a part of the ILP housing may, for example, serve as counter electrode.

With regard to the present invention, the processing unit is generally regarded as a functional unit of the cardiac pacing device, that interprets and executes instructions comprising an instruction control unit and an arithmetic and logic unit. The processing unit may comprise a microprocessor, a controller, a digital signal processor (DSP), an application specific integrated circuit (ASIC), a field-programmable gate array (FPGA), discrete logic circuitry or any combination thereof. Alternatively or additionally, the processing unit may be realized using integrated dedicated hardware logic circuits, in particular in the case of an ILP due to the small size and extreme power limitation. Additionally, the processing unit may be configured to process signal data received from the detector, in particular measured signals of the atrial activity. The processing unit may further comprise a counter and a clock. The counter may be used to count clock signals of the clock. The counter may be started at each sensed atrial or ventricular contraction and count the number of clock signals until the next atrial or ventricular contraction occurs or atrial pacing is provided by the pacing signal generator.

In one embodiment of the cardiac pacing device, the first electrical signal is configured to effectively pace the myocardium based on at least one pre-defined treatment parameter stored in a data memory connected with the pacing signal generator, wherein the second electrical signal has a lower amplitude and/or a lower signal width (signal duration) and/or a different signal form. The starting time point of the second electrical signal relates to the intrinsic contraction event of the chamber. Accordingly, the cardiac pacing device may stimulate at different amplitudes, e.g., in the atrium of the patient's heart (e.g., a real “pacing amplitude”—the first electrical signal—to capture the myocardium and a much lower “trigger amplitude”—the second electrical signal—that only serves to support the far-field sensing), the longevity impact on the device by the AAT stimulation will be reduced. For example, the amplitude of the second electrical signal is at least 20% lower, preferably at least 40% lower, than the amplitude of the first electrical signal. The amplitude of the second electrical signal may lie between 0.01 V and 2 V, for example. Accordingly, alternatively or additionally, the signal width of the second electrical signal may be at least 20% lower or at least 20% higher than the signal width of the first electrical signal, wherein the pulse width of the second electrical signal may be between 0.01 ms and 0.25 ms, preferably between 0.01 ms and 0.1 ms. As also indicated below, the first electrical signal and/or the second electrical signal may be a pulse signal, so that the previously mentioned signal widths in this case are regarded as pulse widths. Additionally or alternatively, the signal form of the first electrical signal and of the second electrical signal may be different. For example, the first electrical signal may be one pulse, whereas the second electrical signal may be at least two pulses (e.g., double or triple pulses) and/or vice versa. In another embodiment, the second electrical signal may be delivered by the pacing signal generator with a pre-defined (short) delay after beginning of the sensed intrinsic atrial event. For example, the second electrical signal is delivered with a delay of at least 0.1 ms after the detection of the sensed intrinsic atrial event thereby ensuring that the second electrical signal is detected in a timely manner to ventricle pacing. This delay should not exceed 100 ms so that the AV-sequential timing can still be ensured and that ventricular pacing is still delivered during the atrial refractory period.

In one embodiment of the cardiac pacing device, the first electrical signal of the cardiac pacing signal is a first pulse signal and the second electrical signal is a second pulse signal, wherein, for example, the second electrical signal is a pulse signal consisting of several pulses provided subsequently. Using a pulse (or a pulsed signal) for pacing the myocardium is, generally, well known. However, according to the present invention, the pacing signal generator provides different types of pacing signals which can be distinguished by a respective detector or processing unit thereby transmitting an information according to the actual state of the actual intrinsic atrial activity. For example, the first pulse signal and the second pulse signal may be a rectangular pulse, a cosine squared pulse, a Gaussian pulse or similar. For pulsed first and second signals the above explained differences in the signal parameters may apply analogously.

In one embodiment of the cardiac pacing device, the detector is configured to capture time-dependent electromagnetic signals and/or electric signals and/or sound signals of the atrial activity and, if applicable, of the ventricular activity. For example, the cardiac pacing device detects time-dependent electrical depolarization and repolarization field signals such as an electrocardiogram (ECG) or an IEGM (intracardiac electrogram) within the patient's heart using, for example, the at least one electrode, e.g., within the atrium of the patient's heart (i.e., an atrial IEGM), for example a high resolution IEGM. The ECG or IEGM signal reflects and comprises, for example, the heart rate, PR-interval, QT-interval, ST-interval, P-wave and T-wave durations. From the signals reflecting the heart's activity, in particular the atrial activity, the detector may derive electrical signals such as electrical signals associated with the contraction of one of the atria (in the following intrinsic atrial signal) and electrical signals associated with the contraction of the ventricles (in the following intrinsic ventricular signal). In the case of an ILP located within a ventricle, the intrinsic atrial signal may be a far field signal. The detector may further be configured to differentiate between intrinsic atrial signals and intrinsic ventricular signals. Further, the detector of the cardiac pacing device additionally senses a pacing signal (e.g., its start time position and its duration as well as its rate or frequency) from another cardiac pacing device. These detected time-dependent signals may be filtered and/or amplified and transmitted to the processing unit. The processing unit may then detect from the received signal whether an intrinsic atrial event is present or not based on this signal, e.g., on the presence or absence of a spontaneous p-wave within the atrial IEGM. If there is no spontaneous p-wave within a pre-defined time period from the beginning of the respective cardiac cycle, the processing unit triggers the pacing signal generator to directly provide the first electrical signal to pace the atrium without further delay. The time point of pacing depends on the time period at which the pacing signal generator triggers pacing. If a spontaneous p-wave is detected, the processing unit triggers the pacing signal generator to provide the second electrical signal after a pre-defined second time period running from the start of the spontaneous p-wave.

The cardiac pacing device may comprise a data memory which may include any volatile, non-volatile, magnetic, or electrical media, such as a random access memory (RAM), read-only memory (ROM), non-volatile RAM (NVRAM), programmable ROM (e.g., EEPROM), flash memory, or any other memory device. The data memory saves the above and below mentioned thresholds, values, parameters (e.g., parameters of the first, second and third signal) and conditions. They are required by the processing unit during processing the above and below explained steps.

The detector may comprise additionally to the above functions an accelerometer, a vibration sensor, an acoustic sensor (including ultrasound) and/or any other mechanical, electric and/or magnetic sensor that is capable to detect the actual activity of the patient dependent on time (i.e., a motion sensor), e.g., whether the patient moves or doesn't move, for example changes body position/posture, sleeps, sits, moves fast or slowly, including exercising. The detector collects the activity signals of the patient and transforms them into electrical signals. Further, the cardiac pacing device may comprise a communication unit comprising a transceiver in order to exchange data with an external device such as a computer or a programmer.

Generally, the detector and/or the processing unit may digitize all or some detected or measured analog signals, filter them and/or smooth them in order to reduce signal noise and/or cull specific metrics. Some pre-processing steps may be provided by the detector, as well.

The units and components of the cardiac pacing device may be contained within a hermetically sealed housing.

At least the above problem is further solved by a system comprising the above described cardiac pacing device as a first cardiac pacing device comprising a first processing unit, a first detector and a first pacing signal generator, wherein the system further comprises a second cardiac pacing device, wherein the second cardiac pacing device comprises a second processing unit for controlling a second pacing signal generator and further comprises a second detector configured to measure a time-dependent signal of a heart's activity. Further, the second processing unit is configured to identify from the signal of the heart's activity received from the second detector a pacing signal and whether this pacing signal corresponds to the first electrical signal or to the second electrical signal provided by the first cardiac pacing device, wherein the second processing unit is configured to control the second pacing signal generator such that it provides a third electrical signal if ventricular pacing is intended within the respective cardiac cycle, wherein the third electrical signal realizes at least one pre-defined signal parameter value that depends on whether the pacing signal corresponds to the first electrical signal or to the second electrical signal was previously identified within the same cardiac cycle. The first cardiac pacing device and the second cardiac pacing device may comprise the modules, elements and units described above with regard to the cardiac pacing device.

In one embodiment, the first cardiac pacing device may be an atrial ILP and the second cardiac pacing device a ventricular ILP. The first cardiac pacing device paces the atrium of the patient's heart by the first electrical signal or administers the second electrical signal as indicated above in case an intrinsic atrial event is detected. The second detector of the second cardiac pacing device then detects the respective electrical signal close to the atrial stimulus which was triggered by the first electrical signal or caused by the heart's internal processes.

Accordingly, if the second cardiac pacing device (operated in, for example, the VDD mode) intends to pace the ventricle, its second pacing signal generator provides a third electrical signal, wherein its parameter depend on whether previously a first or a second electrical signal was provided by the first cardiac pacing device. For example, the starting time point of the third electrical signal depends on whether the first electrical signal or the second electrical signal was detected within the respective cardiac cycle before (sense compensation). Generally, the second electrical signal may occur later than the first electrical signal with regard to the intrinsic atrial event. Accordingly, the time difference of the second electrical signal and the third electrical signal may be shorter than the time difference of the first electrical signal and the third electrical signal.

The first processing unit, first pacing signal generator, first detector and, if applicable, a first data memory, are all electrically connected and contained within the housing of the first cardiac pacing device. The second processing unit, second pacing signal generator, the second detector and, if applicable, a second data memory, are all electrically connected and contained within the housing of the second cardiac pacing device. The first processing unit and/or the second processing unit may comprise hardware to support signal processing (e.g., scaling, filtering, rectification) of the signals received from the first detector and the second detector, respectively.

In one embodiment, the second cardiac pacing device may be operated in an inhibited mode with regard to the ventricular intrinsic event (or ventricular demand mode), for example an VDD mode, which means that the second processing unit is further configured to detect a presence and absence of an intrinsic ventricular event from the signal of the heart's activity received from the second detector and to provide the third electrical signal if absence of the intrinsic ventricular event is detected within the respective cardiac cycle. The presence or absence of the intrinsic ventricular event is detected within a pre-defined time period from the beginning of the respective cardiac cycle, for example within the so-called AV delay. In one example, the system comprising the first cardiac pacing device and the second cardiac pacing device realizes dual-chamber pacing, e.g., using a DDD mode, wherein the first cardiac pacing device realizes an AAT mode and the second cardiac pacing device a VDD mode. In one embodiment, The AAT mode may additionally be limited to a pre-defined atrial rate, i.e., around an upper rate threshold for tracking, if the support of the detection of the intrinsic atrial event (p-wave detection) in the ventricular VDD device is not required or desired at high atrial rates. Alternatively, the second cardiac pacing device may operate in the VVI or the VVI-R mode.

The above system has the advantages described above with regard to the cardiac pacing device. Further, it is advantageous that the first cardiac pacing device and the second cardiac pacing device do not need to be compatible with regard to their communication type and communication channel. The pacing of the second cardiac pacing device is more reliable and the devices may better remain synchronized with each other, wherein the pacing is adapted to the time of the atrial contraction, because the first and the second signals are better detectable than the intrinsic atrial event (p-wave) detected from a location different from the atrium, namely, from the ventricle.

At least the above problem is further solved by a method of operating a cardiac pacing device for implantation in a patient's heart, wherein the cardiac pacing device comprises a processing unit for controlling a pacing signal generator and further comprises a detector for measuring a time-dependent signal of an intrinsic atrial activity, wherein the processing unit detects a presence and absence of an intrinsic atrial event within a cardiac cycle based on the measured signal of the atrial activity within the respective cardiac cycle received from the detector, wherein the processing unit controls the pacing signal generator such that it provides a first electrical signal in case of absence of the intrinsic atrial event within the respective cardiac cycle and a second electrical signal in case of presence of the intrinsic atrial event within the respective cardiac cycle, wherein the second electrical signal is different from the first electrical signal.

In one embodiment of the method of operating the cardiac pacing device, the first electrical signal effectively paces the myocardium based on at least one pre-defined treatment parameter, wherein the second electrical signal has a lower amplitude and/or a lower electrical signal width and/or a different electrical signal form compared to the first electrical signal.

In one embodiment of the method of operating the cardiac pacing device, the first electrical signal is a first pulse signal and the second electrical signal is a second pulse signal, wherein, for example, the second electrical signal is a pulse signal consisting of several pulses provided subsequently.

In one embodiment of the method of operating the cardiac pacing device, the detector captures time-dependent electromagnetic signals and/or electric signals and/or sound signals of the atrial activity.

At least the above problem is further solved by an operation method of a system comprising the cardiac pacing device described above as a first cardiac pacing device comprising a first processing unit, a first detector and a first pacing signal generator, wherein the system further comprises a second cardiac pacing device, wherein the second cardiac pacing device comprises a second processing unit for controlling a second pacing signal generator and further comprises a second detector for measuring a time-dependent signal of a heart's activity, wherein the second processing unit identifies from the signal of the heart's activity received from the second detector a pacing signal and whether this pacing signal corresponds to the first electrical signal or to the second electrical signal provided by the first cardiac pacing device, wherein the second processing unit controls the second pacing signal generator such that it provides a third electrical signal if ventricular pacing is intended within the respective cardiac cycle, wherein the third electrical signal realizes at least one pre-defined signal parameter value that depends on whether the pacing signal corresponds to the first electrical signal or to the second electrical signal was previously identified within the same cardiac cycle.

In one embodiment of the method of operating the system, the second processing unit detects a presence and absence of an intrinsic ventricular event from the signal of the heart's activity received from the second detector and provides the third electrical signal if absence of the intrinsic ventricular event is detected within the respective cardiac cycle.

The above embodiments of the operation method of the cardiac pacing device and the system have the same advantages as described above with regard to the cardiac pacing device and the system. Embodiments of the cardiac pacing device and the system, respectively, indicated above may be realized in the respective operation method analogously. It is referred to the above explanation of the cardiac pacing device and of the system in this regard.

Each one of the above methods is, for example, realized as a computer program which comprises instructions which, when executed, cause the processing unit (processor) to perform the steps of the above method (to be executed by the medical device, in particular at its processor) which is a combination of above and below specified computer instructions and data definitions that enable computer hardware to perform computational or control functions or which is a syntactic unit that conforms to the rules of a particular programming language and that is composed of declarations and statements or instructions needed for an above and below specified function, task, or problem solution.

Furthermore, a computer program product is disclosed comprising instructions which, when executed by the processing unit, cause the processing unit to perform the steps of the above defined method. Accordingly, a computer readable data carrier storing such computer program product is disclosed.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawing, wherein

FIG. 1 shows a first embodiment of a system of an atrial ILP and a ventricular ILP within a cross section of a patient's heart,

FIG. 2 depicts a functional block diagram of the atrial ILP shown in FIG. 1,

FIG. 3 depicts a functional block diagram of the ventricular ILP shown in FIG. 1,

FIG. 4 shows IEGM signals of the atrial ILP and the ventricular ILP of FIG. 1 over time in the case that no intrinsic atrial event is detected and therefore an atrial pace is delivered, and

FIG. 5 shows IEGM signals of the atrial ILP and the ventricular ILP of FIG. 1 over time in the case that an intrinsic atrial event is detected.

### DETAILED DESCRIPTION

In the following, the present invention is described with regard to a dual-chamber ILP system comprising an atrial ILP and a ventricular ILP. It may analogously be realized in a system comprising a defibrillator or another device which has a cardiac pacing function, as well. Additionally, the atrial ILP is an embodiment for an above-described cardiac pacing device.

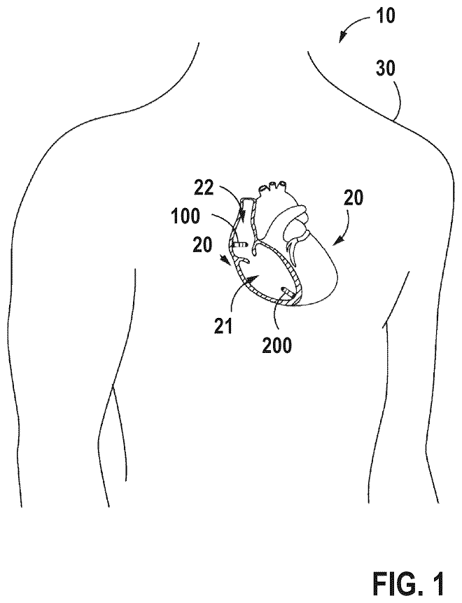

FIG. 1 shows an example leadless pacing system 10 implanted within the heart 20 of a patient 30. Leadless pacing system 10 includes an atrial ILP 100 and a ventricular ILP 200. Atrial ILP 100 may be configured to be implanted within the right atrium 22 of the heart and pace this atrium, sense intrinsic atrial depolarizations, and inhibit atrial pacing in response to detected atrial depolarization. Ventricular ILP 200 may be implanted within the right ventricle 21 of the heart 20 and configured to monitor the electrical activity of the heart 20, sense atrial and ventricular depolarizations (e.g., intrinsic atrial and ventricular depolarizations), and inhibit ventricular pacing in response to detected ventricular depolarization. A programmer (not shown) may be used to program atrial ILP 100 and/or ventricular ILP 200 and retrieve data from atrial ILP 100 and/or ventricular ILP 200.

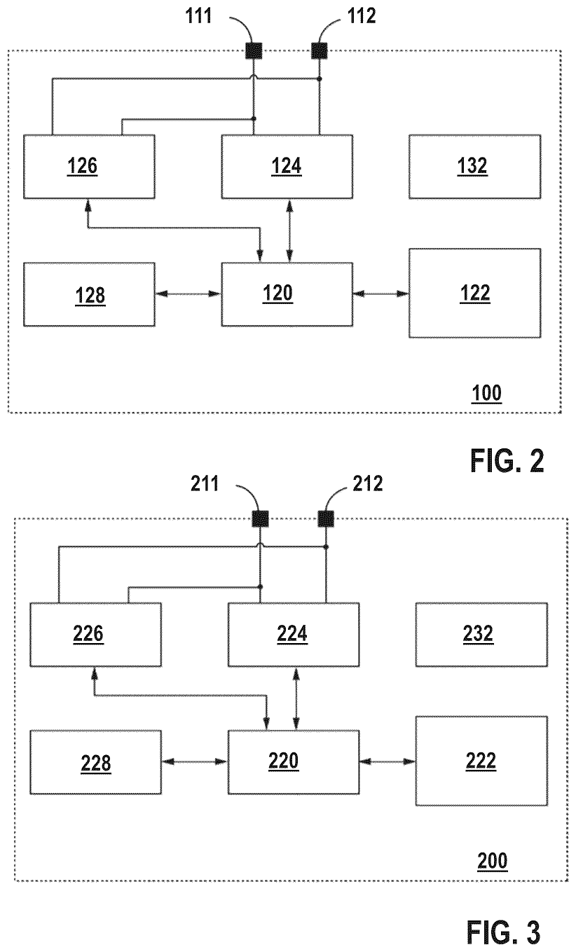

FIG. 2 shows a functional block diagram of the atrial ILP 100 configured for implantation within atrium 22 (FIG. 1). The atrial ILP 100 comprises, for example, a processing unit 120 with a clock, at least one counter for the clock signals and a data memory 122, a pacing signal generator 124, a detector 126, and a power source 132. It also may comprise in one embodiment a communication unit 128. The power source 132 may be electrically connected to one or more of the other components/units 120, 122, 124, 126, 128 (not shown in FIG. 2) and may include a battery, e.g., a rechargeable or non-rechargeable battery. The power source 132 provides electrical energy to all units and components of the ventricular ILP 100 (not explicitly shown in the figure to keep the figure simple), in particular to all units mentioned above and is therefore electrically connected to these units and components. Units included in atrial ILP 100 represent their respective functionality. Similar or identical units and functionality may also be included in the atrial ILP 100. Units of the atrial ILP 100 of present disclosure may include any discrete and/or integrated electronic circuit components that implement analog and/or digital circuits capable of producing the functions attributed to the units herein. For example, the units may include analog circuits, e.g., amplification circuits, filtering circuits, and/or other signal conditioning circuits. The units may also include digital circuits, e.g., combinational or sequential logic circuits, memory devices, etc. The units may further be realized using integrated dedicated hardware logic circuits. The data memory 122 may include any volatile, non-volatile, magnetic, or electrical media mentioned above. Furthermore, the processing unit 120 may include instructions that, when executed by one or more processing circuits, cause the units to perform various functions attributed to these units herein. The functions attributed to the units herein may be embodied as one or more processors, hardware, firmware, software, or any combination thereof. Depiction of different features as units is intended to highlight different functional aspects, and does not necessarily imply that such units must be realized by separate hardware or software components. Rather, functionality associated with one or more units may be performed by separate hardware or software components, or integrated within common or separate hardware or software components. Data memory 122 may store computer-readable instructions that, when executed by processing unit 120, cause processing unit 120 to perform the various functions attributed to processing unit 120 herein. Further, data memory 122 may store parameters for these functions, e.g., pacing signal parameters, values, conditions and thresholds described above and below. The pacing instructions and pacing signal parameters, conditions and thresholds may be updated by the programmer using the communication unit 128. The communication unit 128 may comprise an antenna, coil, patient anatomical interface, and/or a transceiver.

The processing unit 120 may communicate with pacing signal generator 124 and detector 126 thereby transmitting signals. Pacing signal generator 124 and detector 126 are electrically coupled to electrodes 111, 112 of the ventricular ILP 100. Detector 126 is configured to monitor signals from electrodes 111, 112 in order to monitor electrical activity of heart 20 and to transmits these signals to the processing unit 120. Pacing signal generator 124 is configured to deliver electrical stimulation signals to atrium 22 via electrodes 111, 112, for example a first electrical signal (pacing pulse) Ap and a second electrical signal (pacing pulse) Ap*. Processing unit 120 may control pacing signal generator 124 to generate and deliver this electrical stimulation to atrium 22 via electrodes 111, 112. Processing unit 120 may control pacing signal generator 124 to deliver electrical stimulation therapy using the pacing information described above and below, according to one or more therapy programs including pacing parameters, which may be stored in data memory 122. The pacing signals are produced by the processing unit 120 based on a determined atrial pacing time value and/or the atrial pacing rate value, wherein the atrial pacing time value and/or the atrial pacing rate value is calculated by the processing unit according to the intrinsic cardiac signals and/or is derived from the data memory 122 based on a pre-defined therapy program.

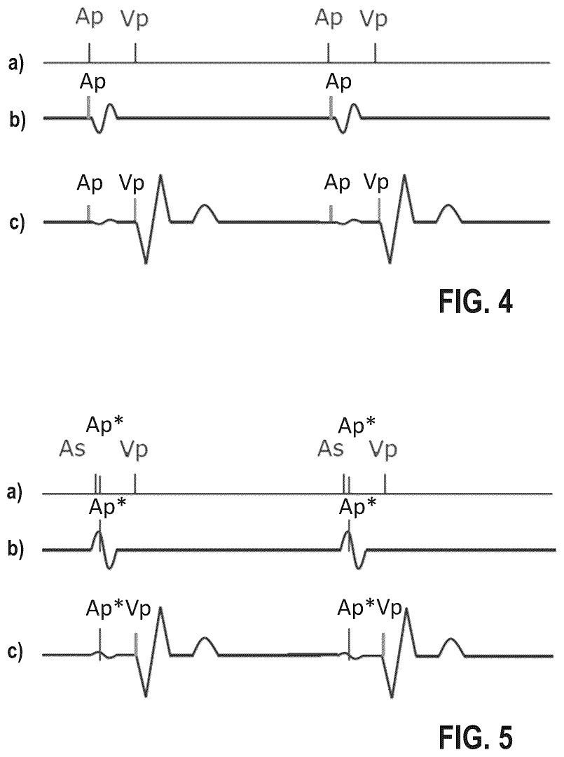

Detector 126 may further include circuits that acquire time-dependent electrical signals (e.g., electric depolarization and repolarization signals, IEGM) from the heart including intrinsic cardiac electrical activity, such as intrinsic atrial events and, if applicable, intrinsic ventricular events. Examples of IEGM signals detected by detector 126 of the atrial ILP 100 are depicted in FIGS. 4b) and 5b), or the line b) in FIGS. 4 and 5). Detector 126 may filter, amplify, and digitize the acquired electrical events of the heart chambers contractions. Processing unit 120 may receive the detected intrinsic atrial events and, if applicable, the intrinsic ventricular events provided by detector 126. Processing unit 120 may assess cardiac activity signals comprising the intrinsic atrial events and, if applicable, the intrinsic ventricular events received from the detector 126 and is configured to determine from the IEGM signals the presence or absence of an intrinsic atrial event (p-wave) within a pre-defined time period from the start of the cardiac cycle within each cardiac cycle.

Atrial ILP 100 may include a housing, anchoring fixation features, and electrodes 111, 112. The housing may have a pill-shaped cylindrical form factor in some examples. The anchoring fixation features are configured to connect atrial ILP 100 to heart 20. Anchoring fixation features may be fabricated from a shape memory material, such as Nitinol. For example, as illustrated and described herein with respect to FIG. 1, anchoring fixation features may be configured to anchor atrial ILP 100 to heart 20 within right atrium 22. Although ILP 10 includes a plurality of anchoring fixation features/elements that are configured to stably engage ILP 10 to cardiac tissue in the right atrium, it is contemplated that a cardiac pacing device according to the present disclosure may be engaged with cardiac tissue in other chambers of a patient's heart 20 using other types of anchoring fixation features.

As indicated above, atrial ILP 100 may include two electrodes 111, 112, although more than two electrodes may be included on a cardiac pacing device in other examples. Electrodes 111, 112 may be spaced apart a sufficient distance to be able to detect various electrical signals generated by the heart 20, such as p-waves generated by the atrium and QRS complex generated by the ventricle. The housing houses electronic components of atrial ILP 100. Electronic components may include any discrete and/or integrated electronic circuit components that implement analog and/or digital circuits capable of producing the functions attributed to atrial ILP 100 described above and below.

As shown in FIG. 3 the ventricular ILP 200 may have basically the same hardware and software structure as the atrial ILP 100 comprising a processing unit 220 with a clock, at least one counter for the clock signals and a data memory 222, a pacing signal generator 224, a detector 226, a power source 232 as well as electrodes 211, 212. It also may comprise in one embodiment a communication unit 228. The power source 232 may be electrically connected to one or more of the other components/units 220, 222, 224, 226, 228 (not shown in FIG. 3). Accordingly, it is referred to the above explanation of the elements and modules with regard to the atrial ILP 100. However, the ventricular ILP 200 is configured to pace the right ventricle 21 of the patient's heart 20. Accordingly, the pacing parameters and treatment program(s) stored in the data memory 222 are adapted to this function of the ventricular ILP 200. Further, the detector 226 may include circuits that acquire time-dependent electrical signals (e.g., electric depolarization and repolarization signals, IEGM) from the heart including intrinsic cardiac electrical activity, such as intrinsic atrial events, atrial pacing signals and intrinsic ventricular events. Examples of such IEGM are depicted in FIGS. 4c) and 5c), or the line c) in FIGS. 4 and 5. Detector 226 may filter, amplify, and digitize the IEGM signals. Processing unit 220 may receive the detected intrinsic ventricular events, the intrinsic atrial events and atrial pacing signals provided by detector 226. Processing unit 220 may assess cardiac activity signals received from the detector 226 and is configured to determine from the IEGM signals the presence or absence of an intrinsic ventricular event within a pre-defined time period from the start of the cardiac cycle within each cardiac cycle.

The system 10 shown in FIG. 1 comprising the atrial ILP 100 and the ventricular ILP 200 realizes an improved DDD mode, wherein the atrial ILP 100 operates according to a modified AAT mode and the ventricular ILP according to a VDD mode. This is explained in the following with regard to IEGM signals shown in FIGS. 4 and 5. The line a) in FIGS. 4 and 5 show only the pacing pulses Ap, Ap* and Vp provided by atrial ILP 100 and ventricular ILP 200, respectively.

In order to reduce the energy consumption by the AAT mode in the atrial ILP 100 realizes the modified AAT mode and provides a “normal” atrial stimulus (first electrical signal mentioned above, Ap in FIG. 4) in case of absence of an intrinsic atrial event (FIG. 4) and a stimulus at a much lower amplitude and potentially further different characteristics (second electrical signal mentioned above, Ap* in FIG. 5) in case of presence of an intrinsic atrial event (As, FIG. 5). The absence of an intrinsic atrial event is observed by processing unit 120 of atrial ILP 100 by absence of the p-wave within a pre-defined time period from the beginning of the respective cardiac cycle. If the intrinsic atrial event is absent the processing unit 120 triggers the pacing signal generator 124 to provide a pacing signal (first electrical signal) Ap to stimulate atrial contraction as shown in FIG. 4b). The amplitude is dependent on the pacing threshold but expected to lie between 0.5V and 5V (usually around 1-2V), furthermore a pulse width of 0.25-1 ms (default 0.25 ms) and a single pulse (“normal pacing pulse”). Accordingly, in the IEGM of the ventricular ILP 200 which is depicted in FIG. 4c) this electrical signal Ap is contained or may be detected. The processing unit 220 of ventricular 200 assesses the Ap signal and concludes by the detected parameter of this signal that it is a first electrical signal that has provided a “normal” atrial stimulus. Accordingly, the processing unit 220 controls the pacing signal generator 224 to provide a ventricular stimulus Vp (third electrical signal) if it is not inhibited by an intrinsic ventricular event. This is depicted in FIG. 4c).

In case the processing unit 120 observes an intrinsic atrial event As as shown in FIG. 5b), the processing unit 120 controls the pacing signal generator 124 such that it provides a second electrical signal Ap* at a pre-defined time point after detection of the intrinsic atrial event As, e.g., 0.5 ms after detection of As (see FIG. 5b)) but up to 150 ms after detection of As, preferably 5 ms after detection of As. As indicated above, the delivered triggered impulse Ap* may have comparable characteristics as the pacing stimulus Ap, i.e., amplitude between 0.01 V (or even lower) up to the normal amplitude (2 V), preferably an amplitude of 0.5 V, pulse width of 0.01 ms up to the default pulse width of the device (0.25 ms), preferably a pulse width of 0.1 ms, double/triple pulses, or any other pulse pattern that allows simple and highly reliable detection of the second electrical signal Ap* and differentiation from the first electrical signal Ap by the ventricular ILP 200, in particular by its detector 226 and its processing unit 220. Such pulse pattern may consist of pulses of different number (1-x pulses) and alternatively or additionally different amplitude and/or pulse width, so that the single pulses of the second electrical signal Ap* may have identical as well as different characteristics compared with the first electrical signal Ap. The different characteristic of the second electrical signal Ap* is detected by the ventricular ILP 200 and the ventricular pacing signal (third electrical signal) Vp is adapted accordingly (see FIG. 5c)), wherein the adapted ventricular pacing signal Vp is provided if not inhibited by an intrinsic ventricular event. The ventricular pacing signal may be, for example, shifted in time because the detected electrical signal Ap* is provided shortly after beginning of the corresponding intrinsic atrial event As (sense compensation).

Generally, in this embodiment the second processing unit 220 is adapted to control pacing of the right ventricle 21 using the known VDD mode based on received cardiac activity signals. The counter of the processing unit 220 used to time the AV delay (for providing the ventricular pace signal) may also be used to measure intrinsic AV delays. The VDD pacing mode may be R-Sync in the ventricular ILP 200. This means that every cycle is synchronized by every used ventricular event (intrinsic ventricular contraction or ventricular pacing). It is also an atrial tracking mode with high reliability by the first and second electrical signals Ap and Ap* since these signals have a higher amplitude than the intrinsic atrial event As or the paced atrial contraction (see FIGS. 4c) and 5c)). The VDD mode is provided such that every sensed atrial contraction can shift the timing. In other words, VDD is both R-Sync and P-Sync. The timing of the next potential ventricular pacing signal is scheduled based on the most recent ventricular event and a targeted pacing interval (determined from a target cardiac rate).

The cardiac rate is determined from the intrinsic atrial event and, if applicable, from the intrinsic ventricular event.

The lowered amplitude or duration of the signal provided by the atrial ILP 100 reduces the longevity impact of the triggered pacing. Further, the different characteristics of the electrical signals Ap and Ap*, allows the ventricular VDD device to differentiate between an atrial pace (first electrical signal) Ap and a triggered atrial stimulus (second electrical signal) Ap* to adapt the timing of ventricular pacing by the ventricular ILP 200 accordingly, e.g., provide a sense compensation for AV delay. The present invention realizes the modi AAT (modified)+VDD and results in an improved DDD mode with less longevity impact and size reduction.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A cardiac pacing device for implantation in a patient's heart, wherein the cardiac pacing device comprises a processing unit for controlling a pacing signal generator and further comprises a detector configured to measure a time-dependent signal of an intrinsic atrial activity, wherein the processing unit is configured to detect a presence and absence of an intrinsic atrial event within a cardiac cycle based on the measured signal of the atrial activity within the respective cardiac cycle received from the detector, wherein the processing unit is configured to control the pacing signal generator such that it provides a first electrical signal in case of absence of the intrinsic atrial event within the respective cardiac cycle and a second electrical signal in case of presence of the intrinsic atrial event within the respective cardiac cycle, wherein the second electrical signal is different from the first electrical signal.

2. The cardiac pacing device of claim 1, wherein the first electrical signal is configured to effectively pace the myocardium based on at least one pre-defined treatment parameter, wherein the second electrical signal has a lower amplitude and/or a lower signal width and/or a different signal form compared to the first electrical signal.

3. The cardiac pacing device of claim 1, wherein the first electrical signal is a first pulse signal and the second electrical signal is a second pulse signal, wherein, for example, the second electrical signal is a pulse signal consisting of several pulses provided subsequently.

4. The cardiac pacing device of claim 1, wherein the detector is configured to capture time-dependent electromagnetic signals and/or electric signals and/or sound signals of the atrial activity.

5. A system comprising the cardiac pacing device of claim 1, as a first cardiac pacing device comprising a first processing unit, a first detector and a first pacing signal generator, wherein the system further comprises a second cardiac pacing device, wherein the second cardiac pacing device comprises a second processing unit for controlling a second pacing signal generator and further comprises a second detector configured to measure a time-dependent signal of a heart's activity, wherein the second processing unit is configured to identify from the signal of the heart's activity received from the second detector a pacing signal and whether this pacing signal corresponds to the first electrical signal or to the second electrical signal provided by the first cardiac pacing device, wherein the second processing unit is configured to control the second pacing signal generator such that it provides a third electrical signal if ventricular pacing is intended within the respective cardiac cycle, wherein the third electrical signal realizes at least one pre-defined signal parameter value that depends on whether the pacing signal corresponds to the first electrical signal or to the second electrical signal was previously identified within the same cardiac cycle.

6. The system of claim 5, wherein the second processing unit is further configured to detect a presence and absence of an intrinsic ventricular event from the signal of the heart's activity received from the second detector and to provide the third electrical signal if absence of the intrinsic ventricular event is detected within the respective cardiac cycle.

7. The system of claim 5, wherein the first cardiac pacing device is an atrial ILP and the second cardiac pacing device is a ventricular ILP.

8. A method of operating a cardiac pacing device for implantation in a patient's heart, wherein the cardiac pacing device comprises a processing unit for controlling a pacing signal generator and further comprises a detector for measuring a time-dependent signal of an intrinsic atrial activity, wherein the processing unit detects a presence and absence of an intrinsic atrial event within a cardiac cycle based on the measured signal of the atrial activity within the respective cardiac cycle received from the detector, wherein the processing unit controls the pacing signal generator such that it provides a first electrical signal in case of absence of the intrinsic atrial event within the respective cardiac cycle and a second electrical signal in case of presence of the intrinsic atrial event within the respective cardiac cycle, wherein the second electrical signal is different from the first electrical signal.

9. The method of claim 8, wherein the first electrical signal effectively paces the myocardium based on at least one pre-defined treatment parameter, wherein the second electrical signal has a lower amplitude and/or a lower electrical signal width and/or a different electrical signal form compared to the first electrical signal.

10. The method of claim 8, wherein the first electrical signal is a first pulse signal and the second electrical signal (Aps is a second pulse signal, wherein, for example, the second electrical signal is a pulse signal consisting of several pulses provided subsequently.

11. The method of claim 8, wherein the detector captures time-dependent electromagnetic signals and/or electric signals and/or sound signals of the atrial activity.

12. An operation method of a system comprising the cardiac pacing device of claim 1, as a first cardiac pacing device comprising a first processing unit, a first detector and a first pacing signal generator, wherein the system further comprises a second cardiac pacing device, wherein the second cardiac pacing device comprises a second processing unit for controlling a second pacing signal generator and further comprises a second detector for measuring a time-dependent signal of a heart's activity, wherein the second processing unit identifies from the signal of the heart's activity received from the second detector a pacing signal and whether this pacing signal corresponds to the first electrical signal or to the second electrical signal provided by the first cardiac pacing device, wherein the second processing unit controls the second pacing signal generator such that it provides a third electrical signal if ventricular pacing is intended within the respective cardiac cycle, wherein the third electrical signal realizes at least one pre-defined signal parameter value that depends on whether the pacing signal corresponding to the first electrical signal or to the second electrical signal was previously identified within the same cardiac cycle.

13. The method of claim 12, wherein the second processing unit detects a presence and absence of an intrinsic ventricular event from the signal of the heart's activity received from the second detector and provides the third electrical signal if absence of the intrinsic ventricular event is detected within the respective cardiac cycle.

14. A computer program product comprising instructions which, when executed by a processing unit, cause the respective processing unit to perform the steps of the method according to claim 8.

15. Computer readable data carrier storing a computer program product according to claim 14.
